(19) United States
(12) Patent Application Publication
(10) Pub. No.: US 2010/0028893 A1
(43) Pub. Date: Feb. 4, 2010

(54) METHODS AND COMPOSITIONS FOR PHARMACOGENETIC ANALYSIS OF ANTI-INFLAMMATORY DRUGS IN THE TREATMENT OF RHEUMATOID ARTHRITIS AND OTHER INFLAMMATORY DISEASES

(75) Inventors: Kenneth S. Kornman, Newton, MA (US); Kenneth Huttner, Chestnut Hill, MA (US); John Rogus, North Andover, MA (US); Venkateswarlu Kondragunta, Woburn, MA (US); Xiaodong Wu, Needham, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: INTERLEUKIN GENETICS, INC., Waltham, MA (US)

(21) Appl. No.: 12/504,961

(22) Filed: Jul. 17, 2009

Related U.S. Application Data
(60) Provisional application No. 61/081,937, filed on Jul. 18, 2008.

(51) Int. Cl. C12Q 1/68

(52) U.S. Cl. 435/6

(57) ABSTRACT

The invention provides methods and compositions for the pharmacogenetic analysis of anti-inflammatory compounds, especially for the pharmacogenetic association of responsiveness to rheumatoid arthritis medications that target TNFα.

### RELATED APPLICATIONS

This application claims the benefit of the filing date of U.S. Provisional Patent Application No. 61/081,937, filed on Jul. 18, 2008, the contents of which are incorporated herein by reference in its entirety.

### BACKGROUND OF THE INVENTION

Rheumatoid arthritis (RA) is a chronic disease of unknown cause characterized by prolonged inflammation, swelling and pain of multiple joints. With time, the chronic inflammation leads to bone destruction within the joints and to progressive disability. One prominent hallmark of rheumatoid arthritis is wide variability in its clinical presentation. This variability extends to the level of pain, number of swollen joints and extent of joint deformity. Similarly, the response of patients with rheumatoid arthritis to any specific medical therapy also varies widely, from near elimination of disease signs and symptoms in some patients, to almost complete unresponsiveness in others. Although the underlying cause of the variable clinical expression is not entirely known, results of several studies indicate that differences in individual genetic factors play a central role.

Inflammation in rheumatoid arthritis involves the action of several proteins in the body known as inflammatory cytokines. Among the cytokines, two can be viewed as truly pivotal since differences in the level of their activity largely determine differences in the overall disease severity. These two cytokine proteins are referred to as IL-1 (for interleukin-1) and TNFα (for Tumor Necrosis Factor Alpha). Within an arthritic joint, both IL-1 and TNFα are active and critical for producing the clinical signs and symptoms of the disease. Perhaps more importantly, these two cytokines interact at several biologic levels, with each exerting a pronounced regulatory effect on the other.

Recently, a series of protein-based drugs that act by blocking IL-1 or TNFα activity have received regulatory approval for the treatment of rheumatoid arthritis. Four members of this new class of anti-cytokine drugs, entancercept (Enbrel®), infliximab (Remicade®), anakinra (Kineret®), and more recently adalimumab (Humira®), are on the market in the United States. Enbrel®, Remicade® and Humira® exert their effects by blocking TNFα action, while Kineret® is a selective blocker of IL-1 biological activity. Although these new protein-based compounds have an average efficacy/safety profile superior to the older, small molecule drugs, they have failed to show efficacy in 30-53% of patients studied in a variety of controlled clinical trials. One explanation put forth to account for the variation in responses to each compound is that some individuals with rheumatoid arthritis likely have a more dominant contribution to their disease from IL-1, while in others TNFα activity dominates.

In addition to the incomplete efficacy of these new biologic agents, their expanded use in the rheumatoid arthritis population has raised serious concerns of associated risks of rare but serious adverse events. A report has shown an increased rate of tuberculosis among individuals treated with Remicade®. Remicade® exposure was associated with a 4-fold risk of any active tuberculosis, and a 10-fold risk of extra-pulmonary dissemination.

Despite failing to provide clinical benefit to many patients with rheumatoid arthritis while at the same time increasing the risk of adverse effects, these therapies are also quite expensive. Annual treatment costs generally exceed $10,000. Given the episodic nature of disease progression in RA, many patients may be treated with an ineffective new therapy for several months before the drug failure is recognized by the patient and the physician. Therefore, a simple and inexpensive test to assist physicians and patients in selecting which biologic anti-cytokine therapy would most likely bring clinical benefit would be a clinically valuable tool.

### SUMMARY OF THE INVENTION

The invention provides a genetic predisposition test that identifies subjects that have an increased or decreased chance of responding to anti-TNFα therapy for rheumatoid arthritis.

The invention provides a method for determining a predisposition to efficacious response to rheumatoid arthritis with an anti-TNFα therapy in a subject comprising detecting a pattern selected from: two copies of IL1A (4845) allele T, two copies of IL1B (−511) allele C, and two copies of IL1RN (2018) allele T; two copies of IL1B (−511) allele T, and at least one copy of IL1RN (2018) allele C; wherein the presence of said pattern indicates that said subject is predisposed to efficacious response to an anti-TNFα therapy. Optionally, the anti-TNFα therapy is selected from etanercept, infliximab and adalimumab.

The invention also provides a method for determining a predisposition to a less efficacious response to rheumatoid arthritis with an anti-TNFα therapy in a subject comprising detecting a pattern of one or two copies of IL1B (−3737) allele T.

The invention also provides a method for determining a predisposition to a less efficacious response to rheumatoid arthritis with an anti-TNFα therapy in a subject comprising detecting a pattern of one or two copies of TNFA (−308) allele A.

The invention also provides a method for determining a predisposition to a less efficacious response to rheumatoid arthritis with an anti-TNFα therapy in a subject comprising detecting a pattern of one copy of IL1B (−511) allele T, and one copy of IL1B (−3737) allele T.

The invention also provides a method for determining a predisposition to a less efficacious response to rheumatoid arthritis with an anti-TNFα therapy in a subject comprising detecting a pattern of one copy of IL1B (−511) allele T, one copy of IL1B (−3737) allele T and two copies of IL1RN (2018) allele T.

The invention also provides a method for determining a predisposition to a less efficacious response to rheumatoid arthritis with an anti-TNFα therapy in a subject comprising detecting two or more genetic units where the units are composed of one or two copies of IL1B (−3737) allele T, two copies of IL1RN (2018) allele C and one or two copies of TNFA (−308) allele A.

The invention also provides a method for determining predisposition to differential responses to anti-IL-1 and anti-TNFα therapies in rheumatoid arthritis.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is a bar graph showing the efficacy of Enbrel® and Remicade® treatment in patients with various IL1 genotype patterns.

### DETAILED DESCRIPTION OF THE INVENTION

The invention relates to the improved management of rheumatoid arthritis by genetic analysis of patients to guide the choice and timing of drug treatments for inflammatory disorders by correlating individual responsiveness to drugs with composite genotypes derived from multiple inflammatory genes.

IL-1 and TNFα cytokines are both produced primarily by mononuclear phagocytes and are among the first genes activated in response to infectious agents and antigen-stimulated T-cells, and are mediators of innate immune mechanisms. TNFα is produced in response to, for example, gram-negative bacterial products and is responsible for many of the systemic complications of severe infections. IL-1 production is also induced by such bacterial products (such as LPS) and by other cytokines such as TNFα. Unlike TNFα, IL-1 is also produced by many cell types other than macrophages, such as neutrophils, epithelial cells and endothelial cells. A second wave of inflammatory mediators respond to IL-1 and TNFα, including IL-6, IL-10, IL-1 receptor antagonist (IL-1Ra), and acute phase proteins. Recent studies have provided insights into the inter-regulation of the two cytokine regulatory systems.

Accordingly, the invention rests in part upon the discovery that the IL-1 and TNFα systems are cross-regulated and that polymorphic alleles from one or both loci can affect this cross-regulation thereby leading to a dominance of either IL-1 or TNFα in different individuals. The combination of key genetic variations ultimately affects susceptibility to inflammatory disease as well as to drugs designed to control inflammatory disease, particularly those drugs which specifically target IL-1 or TNFα. In the biologic pathway that leads to the chronic joint inflammation of RA, an initiating event, perhaps infectious or immunologic in origin, provides a pro-inflammatory stimulus. An acute inflammatory cycle follows with the intended goal of eliminating the inciting agent. In this initial response, activation of specific genetic pathways leads to the enhanced synthesis of pro-inflammatory molecules including IL-1, TNF and IL-6, and to anti-inflammatory molecules including IL-10 and IL-1Ra.

Following the acute inflammatory cycle, multiple secondary cycles occur involving activation of genetic pathways and feedback loops that produce variable quantities of both pro-inflammatory molecules, e.g. cytokines IL-1 and TNFα as well as their receptors, and anti-inflammatory molecules, e.g. receptor antagonists, soluble receptors and immunosuppressive peptides. The objective of the integrated system is to clear the initiating agent, activate tissue repair, and re-establish a health-associated equilibrium. It is now appreciated that to prevent the long term consequences in RA, this initial pro-inflammatory response eventually must lead to restoration of a balance between the pro- and anti-inflammatory mediators.

Chronic and destructive joint inflammation may develop if any or a combination of the following occur: a) the initiating agent cannot be cleared, b) if a disease-associated equilibrium is established, c) or if inflammation resolving molecules are not activated.

Health-associated and disease-associated equilibria are integrated composites involving pro- and anti-inflammatory forces. Among the major contributors to this inflammatory composite equilibrium are molecules of the TNF, IL-1, IL-6, IL-4, IL-10 and interferon families, as well as their receptors and intracellular signaling machinery. At multiple levels these molecular families may stimulate or repress each other's synthesis before equilibrium is reached.

The forces acting to maintain a balanced health-associated state are so effective that single gene mutations that are commonly found in the population are unlikely to upset this steady state. This hypothesis is consistent with the lack of epidemiological evidence for single gene mutations producing persistent joint inflammation in RA. It is reasonable, however, to postulate that a “double-hit” occurrence of variations in two or three key genes involved in the inflammatory process may lead to a stable disease-associated equilibrium. It is also likely that multiple disease-associated stable equilibria may be achieved, depending on the specific mix of gene variations that alter pro- and anti-inflammatory elements.

Since the different disease-associated equilibria would be driven by different combinations of gene variations, the level of specific gene and protein expressions in the RA tissues would be expected to be different. Thus, therapeutic agent A may be very effective in equilibrium #1, but may be of limited value in equilibrium #2.

As a consequence of this novel insight into the mechanism for the persistence of inflammation in RA, we propose that for RA patients developing chronic joint inflammation, analysis of their inherited genetic polymorphisms present in multiple inflammatory pathways will describe a pattern (RA profile) that: (1) is consistent with a pro-inflammatory state; (2) identifies the key inflammatory molecules whose aberrant expression has lead to the persistent joint inflammation; and (3) is predictive of the efficacy of individual drug therapies that target specific components of the inflammatory process.

Inflammatory Gene Loci Polymorphisms

The following inflammatory gene polymorphisms were used to demonstrate the validity of the invention: (Table 1).

TABLE 1
A subset of inflammatory gene polymorphisms

| Gene (Polymorphism) | rs | Allele 1 | Allele 2 |
|---|---|---|---|
| IL1A (4845) | rs17561 | G | T |
| IL1B (−511) | rs16944 | C | T |
| IL1B (−3737) | rs4848306 | C | T |
| IL1RN (2018) | rs419598 | T | C |
| IL10 (−1082) | rs1800896 | G | A |
| TNFA (−308) | rs1800629 | G | A |

The TNF alpha locus polymorphisms of the invention include the TNFa microsatellite allele; the TNFb microsatellite allele; the TNFc microsatellite allele; the TNFA −308 polymorphic allele; the TNF −238 polymorphic allele; the TNFA −1031 polymorphic allele; the TNFA −863 polymorphic allele; and the TNFA −857 polymorphic allele. The TNF alpha locus polymorphisms of the invention further include those described in Hajeer and Hutchinson ((2000) Micros Research and Tech 50: 216-228) the contents of which are incorporated herein by reference.

Detection of Alleles

Allelic patterns, polymorphism patterns, or haplotype patterns can be identified by detecting any of the component alleles using any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion). The allele can optionally be subjected to an amplification step prior to performance of the detection step. Preferred amplification methods are selected from the group consisting of: the polymerase chain reaction (PCR), the ligase chain reaction (LCR), strand displacement amplification (SDA), cloning, and variations of the above (e.g. RT-PCR and allele specific amplification). Oligonucleotides necessary for amplification may be selected, for example, from within the metabolic gene loci, either flanking the marker of interest (as required for PCR amplification) or directly overlapping the marker (as in allele specific oligonucleotide (ASO) hybridization). In a particularly preferred embodiment, the sample is hybridized with a set of primers, which hybridize 5′ and 3′ in a sense or antisense sequence to the vascular disease associated allele, and is subjected to a PCR amplification.

An allele may also be detected indirectly, e.g. by analyzing the protein product encoded by the DNA. For example, where the marker in question results in the translation of a mutant protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications.

A general guideline for designing primers for amplification of unique human chromosomal genomic sequences is that they possess a melting temperature of at least about 50° C., wherein an approximate melting temperature can be estimated using the formula Tₘₑₗₜ=[2X(# of A or T)+4X(# of G or C)].

Many methods are available for detecting specific alleles at human polymorphic loci. The preferred method for detecting a specific polymorphic allele will depend, in part, upon the molecular nature of the polymorphism. For example, the various allelic forms of the polymorphic locus may differ by a single base-pair of the DNA. Such single nucleotide polymorphisms (or SNPs) are major contributors to genetic variation, comprising some 80% of all known polymorphisms, and their density in the human genome is estimated to be on average 1 per 1,000 base pairs. SNPs are most frequently biallelic-occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

A variety of methods are available for detecting the presence of a particular single nucleotide polymorphic allele in a subject. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Publication No. WO92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Publication No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For mutations that produce premature termination of protein translation, the protein truncation test (PTT) offers an efficient diagnostic approach (Roest, et. al., (1993) Hum. Mol. Genet. 2:1719-2 1; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Any cell type or tissue may be utilized to obtain nucleic acid samples for use in the diagnostics described herein. In a preferred embodiment, the DNA sample is obtained from a bodily fluid, e.g, blood, obtained by known techniques (e.g. venipuncture) or saliva. Alternatively, nucleic acid tests can be performed on dry samples (e.g. hair or skin). When using RNA or protein, the cells or tissues that may be utilized must express a metabolic gene of interest.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, N.Y.).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

A preferred detection method is allele specific hybridization using probes overlapping a region of at least one allele of a metabolic gene or haplotype and having about 5, 10, 20, 25, or 30 nucleotides around the mutation or polymorphic region. In a preferred embodiment of the invention, several probes capable of hybridizing specifically to other allelic variants of key metabolic genes are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of a metabolic gene or haplotype under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, the allele of a metabolic gene or haplotype is identified by alterations in restriction enzyme cleavage patterns. For example, sample and control DNA is isolated, amplified (optionally), digested with one or more restriction endonucleases, and fragment length sizes are determined by gel electrophoresis.

In yet another embodiment, any of a variety-of sequencing reactions known in the art can be used to directly sequence the allele. Exemplary sequencing reactions include those based on techniques developed by Maxim and Gilbert ((1977) Proc. Natl Acad Sci USA 74:560) or Sanger (Sanger et al (1977) Proc. Nat. Acad. Sci USA 74:5463). It is also contemplated that any of a variety of automated sequencing procedures may be utilized when performing the subject assays (see, for example Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; and Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an allele of a metabolic gene locus haplotype is hybridized to a CDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify a metabolic gene locus allele. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control metabolif locus alleles are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of alleles in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 1 1:238). In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect alleles of a metabolic gene locus haplotype. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Another embodiment of the invention is directed to kits for detecting a predisposition for responsiveness to certain diets and/or activity levels. This kit may contain one or more oligonucleotides, including 5′ and 3′ oligonucleotides that hybridize 5′ and 3′ to at least one allele of a metabolic gene locus or haplotype. PCR amplification oligonucleotides should hybridize between 25 and 2500 base pairs apart, preferably between about 100 and about 500 bases apart, in order to produce a PCR product of convenient size for subsequent analysis.

The design of additional oligonucleotides for use in the amplification and detection of metabolic gene polymorphic alleles by the method of the invention is facilitated by the availability of both updated sequence information from human chromosome 4q28-q31 - - - which contains the human FABP2 locus, and updated human polymorphism information available for this locus. Suitable primers for the detection of a human polymorphism in metabolic genes can be readily designed using this sequence information and standard techniques known in the art for the design and optimization of primers sequences. Optimal design of such primer sequences can be achieved, for example, by the use of commercially available primer selection programs such as Primer 2.1, Primer 3 or GeneFisher (See also, Nicklin M. H. J., Weith A. Duff G. W., “A Physical Map of the Region Encompassing the Human Interleukin-1α, interleukin-1β and Interleukin-1 Receptor Antagonist Genes” Genomics 19: 382 (1995); Nothwang H. G., et al. “Molecular Cloning of the Interleukin-1 gene Cluster: Construction of an Integrated YAC/PAC Contig and a partial transcriptional Map in the Region of Chromosome 2q13” Genomics 41: 370 (1997); Clark, et al. (1986) Nucl. Acids. Res., 14:7897-7914 [published erratum appears in Nucleic Acids Res., 15:868 (1987) and the Genome Database (GDB) project).

In another aspect, the invention features kits for performing the above-described assays. According to some embodiments, the kits of the present invention may include a means for determining a subject's genotype with respect to one or more metabolic gene. The kit may also contain a nucleic acid sample collection means. The kit may also contain a control sample either positive or negative or a standard and/or an algorithmic device for assessing the results and additional reagents and components including: DNA amplification reagents, DNA polymerase, nucleic acid amplification reagents, restrictive enzymes, buffers, a nucleic acid sampling device, DNA purification device, deoxynucleotides, oligonucleotides (e.g. probes and primers) etc.

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

As described above, the control may be a positive or negative control. Further, the control sample may contain the positive (or negative) products of the allele detection technique employed. For example, where the allele detection technique is PCR amplification, followed by size fractionation, the control sample may comprise DNA fragments of the appropriate size. Likewise, where the allele detection technique involves detection of a mutated protein, the control sample may comprise a sample of mutated protein. However, it is preferred that the control sample comprises the material to be tested. For example, the controls may be a sample of genomic DNA or a cloned portion of a metabolic gene. Preferably, however, the control sample is a highly purified sample of genomic DNA where the sample to be tested is genomic DNA.

The oligonucleotides present in said kit may be used for amplification of the region of interest or for direct allele specific oligonucleotide (ASO) hybridization to the markers in question. Thus, the oligonucleotides may either flank the marker of interest (as required for PCR amplification) or directly overlap the marker (as in ASO hybridization).

Information obtained using the assays and kits described herein (alone or in conjunction with information on another genetic defect or environmental factor, which contributes to osteoarthritis) is useful for determining whether a non-symptomatic subject has or is likely to develop the particular disease or condition. In addition, the information can allow a more customized approach to preventing the onset or progression of the disease or condition. For example, this information can enable a clinician to more effectively prescribe a therapy that will address the molecular basis of the disease or condition.

The kit may, optionally, also include DNA sampling means. DNA sampling means are well known to one of skill in the art and can include, but not be limited to substrates, such as filter papers, the AmpliCard™ (University of Sheffield, Sheffield, England S10 2JF; Tarlow, J W, et al., J. of Invest. Dermatol. 103:387-389 (1994)) and the like; DNA purification reagents such as Nucleon™ kits, lysis buffers, proteinase solutions and the like; PCR reagents, such as 10× reaction buffers, thernostable polymerase, dNTPs, and the like; and allele detection means such as the Hinf1 restriction enzyme, allele specific oligonucleotides, degenerate oligonucleotide primers for nested PCR from dried blood.

Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting. Other features and advantages of the invention will be apparent from the following detailed description and claims.

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth.

The term “allele” refers to the different sequence variants found at different polymorphic regions. The sequence variants may be single or multiple base changes, including without limitation insertions, deletions, or substitutions, or may be a variable number of sequence repeats.

The term “allelic pattern” refers to the identity of an allele or alleles at one or more polymorphic regions. Alternatively, an allelic pattern may consist of either a homozygous or heterozygous state at a single polymorphic site. An allelic pattern may consist of the identity of alleles at more than one polymorphic site.

The terms “control” or “control sample” refer to any sample appropriate to the detection technique employed. The control sample may contain the products of the allele detection technique employed or the material to be tested. Further, the controls may be positive or negative controls. By way of example, where the allele detection technique is PCR amplification, followed by size fractionation, the control sample may comprise DNA fragments of an appropriate size. Likewise, where the allele detection technique involves detection of a mutated protein, the control sample may comprise a sample of a mutant protein. However, it is preferred that the control sample comprises the material to be tested. For example, the controls may be a sample of genomic DNA or a cloned portion containing one or more metabolic genes. However, where the sample to be tested is genomic DNA, the control sample is preferably a highly purified sample of genomic DNA.

The phrases “disruption of the gene” and “targeted disruption” or any similar phrase refers to the site specific interruption of a native DNA sequence so as to prevent expression of that gene in the cell as compared to the wild-type copy of the gene. The interruption may be caused by deletions, insertions or modifications to the gene, or any combination thereof.

The term “haplotype” as used herein is intended to refer to a set of alleles that are inherited together as a group (are in linkage disequilibrium) at statistically significant levels (P_{corr}<0.05). As used herein, the phrase “metabolic haplotype” refers to a haplotype of metabolic gene loci.

“Increased risk” refers to a statistically higher frequency of occurrence of the disease or condition in a subject carrying a particular polymorphic allele in comparison to the frequency of occurrence of the disease or condition in a member of a population that does not carry the particular polymorphic allele.

The term “isolated” as used herein with respect to nucleic acids, such as DNA or RNA, refers to molecules separated from other DNAs, or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

“Linkage disequilibrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage disequilibrium”. The cause of linkage disequilibrium is often unclear. It can be due to selection for certain allele combinations or to recent admixture of genetically heterogeneous populations. In addition, in the case of markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns that are comprised of more than one allele, a first allelic pattern is in linkage disequilibrium with a second allelic pattern if all the alleles that comprise the first allelic pattern are in linkage disequilibrium with at least one of the alleles of the second allelic pattern.

The term “marker” refers to a sequence in the genome that is known to vary among subjects.

A “mutated gene” or “mutation” or “functional mutation” refers to an allelic form of a gene, which is capable of altering the phenotype of a subject having the mutated gene relative to a subject which does not have the mutated gene. The altered phenotype caused by a mutation can be corrected or compensated for by certain agents. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs (e.g. peptide nucleic acids) and as applicable to the embodiment being described, single (sense or antisense) and double-stranded polynucleotides.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene.” A specific genetic sequence at a polymorphic region of a gene is an allele. A polymorphic region can be a single nucleotide, the identity of which differs in different alleles. A polymorphic region can also be several nucleotides long.

The term “propensity to disease,” also “predisposition” or “susceptibility” to disease or any similar phrase, means that certain alleles are hereby discovered to be associated with or predictive of a subject's incidence of developing a particular disease (e.g. a vascular disease). The alleles are thus over-represented in frequency in subjects with disease as compared to healthy subjects. Thus, these alleles can be used to predict disease even in pre-symptomatic or pre-diseased subjects.

As used herein, the term “specifically hybridizes” or “specifically detects” refers to the ability of a nucleic acid molecule to hybridize to at least approximately 6 consecutive nucleotides of a sample nucleic acid.

“Transcriptional regulatory sequence” is a generic term used throughout the specification to refer to DNA sequences, such as initiation signals, enhancers, and promoters, which induce or control transcription of protein coding sequences with which they are operably linked.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “mutation-allele” refers to an allele of a gene which, when present in one or two copies in a subject results in increased propensity to a disorder, or phenotype under investigation. There can be several different mutation-alleles, since several different nucleotide changes in a gene may affect the phenotype under study, with a variation in intensity. The term “mutation-allele,” thus refers to an SNP or allele that is associated with high relative risk for a disorder or phenotype under investigation.

### EXAMPLES

### Example 1

In a retrospective, pharmacogenetic study we determined the association between cytokine polymorphisms and the response to anti-TNFα therapy in subjects with rheumatoid arthritis. We tested for statistically significant differences in genotype frequencies between the patients who responded favorably to the anti-TNF therapy (“Responder”) and those who did not respond favorably (“Non-responder”). For the purposes of this study, response was defined by criteria from patient questionnaires as described below.

The sample for this study (N=333) was selected from an insurance claims database by identifying adults with an ICD-9-CM diagnostic code of rheumatoid arthritis listed on physician claims and at least one pharmacy claim for Enbrel® or Remicade® during the previous 12 months.

Inclusion Criteria
Written informed consent.
Age≧18 years
One or more medical claims that list an ICD-9-CM code for rheumatoid arthritis (diagnostic codes 714 through 714.9, inclusive)
One or more pharmacy claims for Enbrel® or Remicade® during the previous 12 months.

DNA was Genotyped for the Following Genetic Markers:
a) IL1A (4845); rs17561; G>T
b) IL1B (−511); rs16944; C>T
c) IL1B (−3737); rs4848306; C>T
d) IL1B (3954); rs1143634; C>T
e) IL1RN (2018); rs419598; T>C
f) TNFA (−308); rs1800629; G>A
g) IL10 (−1082); rs1800896; G>A

Results:

Due to the complexity in assessing improvement in rheumatoid arthritis, the responsiveness to anti-TNFα therapy was defined in four different ways. Association between inflammatory gene polymorphisms and responsiveness to anti-TNFα therapy was analyzed using each of 4 definitions as described below. All definitions of response used in this patent application are based on the American College of Rheumatology's (ACR) definition of improvement in rheumatoid arthritis.

1. Definition of Response #1

Responders were defined as those individuals who 1) received anti-TNFα therapy (Enbrel® or Remicade®) for at least 3 continuous months within the preceding year, 2) answered “a or b” to question 3 and either 5 or 6; and (3) answered “a or b” to two out of five other questions (7, 8, 9, 10, and 11). See questionnaire in example 2. Using this definition of response, there were 300 responders (90%) and 33 non-responders (10%). Females comprised 78% of the study population.

Individuals with a high positive rate of response to anti-TNF treatment for rheumatoid arthritis (97% response rate in this study) were defined by any one of three IL1 genetic patterns: 1) genotype T/T at IL1A (4845), genotype C/C at IL1B (−511) and T/T genotype at IL1RN (2018); 2) genotype T/T at IL1B (−511) and C/C genotype at IL1RN (2018); or 3) genotype T/T at IL1B (−511) and C/T genotype at IL1RN (2018). These genotype patterns identify 13% of the study population and their response rate (97% positive response) to treatment was significantly greater than all other patients (p<0.05).

Five genotype patterns were identified that were associated with a lower rate of response to the anti-TNF drugs. Each genotype pattern shown in Table 2 was associated with a less efficacious response to anti-TNF drugs in this study. Since these genes interact to define different balances between IL-1 and TNFα, we evaluated the role of composite genotypes on drug responsiveness, where the genetic responsiveness is described by a combination of the risk genotypes.

For the purpose of identifying a group with potentially decreased rates of response to rheumatoid arthritis treatment, we define genetically reduced responsiveness as individuals who have genotype C/T at IL1B (−511) and genotype C/T at IL1B (−3737). Thirty-two percent of the study population was scored as reduced responsiveness to treatment (21% treatment failure rate) compared to the genetically responsive group (3% treatment failure rate).

A more targeted reduced responsiveness group can also be defined by the addition of a second risk gene (IL1RN), requiring genotype T/T at IL1RN in addition to genotype C/T at IL1B (−511) and genotype C/T at IL1B (−3737). This subgroup, which comprises 17% of the study population, has a 28% treatment failure rate (statistically significant in comparison to the responsive group). Additional combinations of the risk genotypes shown in Table 2 demonstrate that specific combinations of inflammatory genotypes are predictive of reduced responsiveness to anti-TNF drugs.

TABLE 2
Inflammatory gene polymorphisms associated with lower response
to anti-TNFα therapy-Definition #1.

| | | | | | | Lower |
|---|---|---|---|---|---|---|
| | | | | | | response |
| | | | | | | rate |
| | | | | | | than |
| Risk | IL1B | IL1B | IL1RN | TNFA | IL10 | efficacious |
| Patterns | (−511) | (−3737) | (2018) | (−308) | (−1082) | pattern¹ |
| | C/T | C/T | | | | p < 0.05 |
| | C/T | C/T | T/T | | | p < 0.05 |
| | C/T | C/T | | G/G or G/A | | p < 0.05 |
| | C/T | C/T | | | G/G | p < 0.05 |
| | C/T | C/T | T/T | G/G or G/A | | p < 0.05 |

¹p-value or patients with indicated genetic pattern compared to patients with one of the efficacious patterns consisting of: a) IL1A (4845) = T/T, IL1B(−511) = C/C, and IL1RN (2018) = T/T; b) IL1B (−511) = T/T, and IL1RN (2018) = C/C; and c) IL1B (−511) = T/T and IL1RN (2018) = C/T.

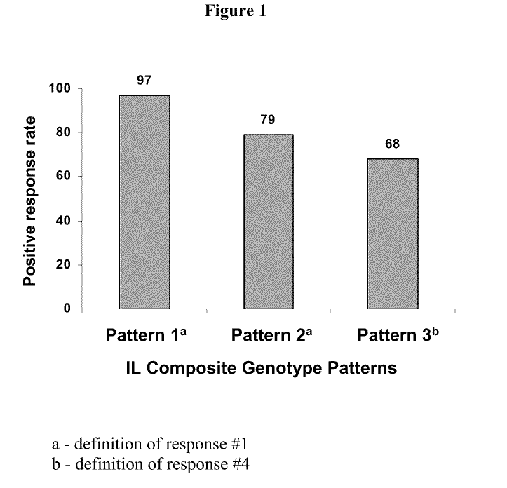

As shown in FIG. 1, patients with one composite genotype pattern demonstrated the highest efficacy with treatment with either Enbrel® or Remicade®. Patients with Pattern 2 (32% of the study populations) had a significantly lower response rate. Patients with Pattern 3, i.e., any 2 risk genetic units at the IL1B (−3737), TNFA (−308) and IL1RN (2018) loci (Table 5) had further reduced response rate with either Enbrel® or Remicade®. These patients comprise 26% of the study population.

2. Definition of Response #2

In the second analysis, more stringent criteria were used to define response to anti-TNFα therapy. Responders were defined as those individuals who 1) received anti-TNFα therapy (Enbrel® or Remicade®) for at least 3 continuous months within the preceding year, 2) answered “a” to questions 5, 6 and 13; and 3) answered “a or b” to question 8. Non-responders were defined as those individuals who 1) answered “b, c, d, e, or f” to questions 5, 6, and 13; and 2) answered “c, d, e, or f” to questions 7, 8, 9, and 10. The rest of the participants were classified as a separate group whose response to anti-TNFα therapy was not clear. See questionnaire in example 2. Using this definition of response, there were 205 responders (73.5%) and 53 non-responders (26.5%).

Under this definition of response, TNFA (−308) was identified as associated with reduced response to anti-TNFα treatment (Table 3). Carriers of the TNFA (−308) genotype A/A or A/G, which comprises 32% of the study population, are 100% less likely to respond to anti-TNFα therapy as compared to carriers of the G/G genotype (OR=2.00 for non-responsiveness, p=0.033).

A trend of association between variations of IL1B (−3737) or IL1RN (2018) and response to anti-TNFα therapy was identified (Table 3). Carriers of T/T or T/C genotype at the IL1B (−3737) locus are 83% more likely to have lower response to anti-TNFα therapy compared to carriers of the IL1B (−3737) C/C genotype (OR=1.83, p=0.099). Carriers of the associated IL1B (−-3737) genotypes comprise 66% of the study population. Similarly, carriers of the IL1RN (2018) genotype C/C are 123% less likely to respond to anti-TNFα therapy, compared to carriers of IL1RN (2018) genotype T/T or T/C (OR=2.23, p=0.108). Carriers of the associated IL1RN (2018) genotype comprise 6% of the study population.

TABLE 3
Inflammatory gene polymorphisms associated with lower response
to anti-TNFα therapy-Definition #2

| | | | | 95% CI | 95% CI | |
|---|---|---|---|---|---|---|
| SNP | Genotype | Frequency | OR | (Low) | (High) | P |
| IL1B (−3737) | T/T, T/C | 0.66 | 1.83 | 0.89 | 3.73 | 0.099 |
| IL1RN (2018) | C/C | 0.06 | 2.23 | 0.84 | 5.92 | 0.108 |
| TNFA (−308) | A/A, A/G | 0.32 | 2.00 | 1.06 | 3.76 | 0.033 |

3. Definition of Response #3.

The third method employed to assess drug response was to treat it as a quantitative trait. A response was measured as the sum of scores for answers for questions 5, 6, 7, 8, 9, 10, and 13. An answer of “a” was assigned a score of 1, a “b” of 2, a “c” of 3, a “d” of 4, a “e” of 5, and a “f” of 6. A greater sum of the scores corresponds to a lower response to the drug.

TABLE 4
Inflammatory gene polymorphisms associated with lower response
to anti-TNFα therapy-Definition #3

| | Associated | | | | |
|---|---|---|---|---|---|
| | allele/genotype | | | | |
| Genetic | IL1B | IL1B | | | |
| marker | (−511) | (−3737) | Frequency | beta | P |
| SNP | | T/T, C/T | 0.66 | 0.10 | 0.033 |
| Haplotype | C | T | 0.42 | 0.07 | 0.061 |
| Diplotype | C/T | C/T | 0.33 | 0.16 | 0.007 |

Under this definition of response, one SNP (IL1B (−3737)) and one diplotype pattern defined by the SNPs IL1B (−511) and IL1B (−3737) were identified as associated with lower response to anti-TNFα treatment (Table 4). Carriers of the IL1B (−3737) genotype T/T or C/T, which comprises 66% of the study population, have lower response as compared to carriers of the C/C genotype (p=0.033). Similarly, carriers of the diplotype pattern, defined by genotype C/T of the IL1B (−511) locus and genotype C/T of the IL1B (−3737) locus, have lower response to anti-TNFα therapy (p=0.007). This group of people comprises 33% of the study population. The reference group consist of individuals with genotype C/C or C/T at the IL1B (−511) locus and C/C at the IL1B (−3737) locus.

We also identified a trend of association between response to anti-TNFα therapy and a haplotype pattern defined by IL1B (−511) and IL1B (−3737) (Table 4). Carriers of the haplotype pattern, defined by allele C of IL1B (−511) and allele T of IL1B (−3737), are more likely to have lower response to anti-TNFα therapy as compared to carriers of the haplotype consisting of IL1B (−511) allele C and IL1B (−3737) allele C (p=0.061). Carriers of the associated haplotype comprise 42% of the study population.

4. Definition of Response #4.

The fourth definition of response is also based on sum of scores. We dichotomized the participants into responders and non-responders based on the sum of scores for answers for questions 5, 6, 7, 8, 9, 10, and 13. Responders were defined as those individuals whose sum of scores is equal or less than 11. Non-responders were defined as individuals with a sum of scores greater than 11. See questionnaire in example 2. Using this definition of response, there were 258 responders (73.5%) and 93 non-responders (26.5%).

TABLE 5
Inflammatory gene polymorphisms associated with lower response to anti-
TNFα therapy-Definition #4

| | Associated allele/genotype | | | | | | | | | |
|---|---|---|---|---|---|---|---|---|---|---|
| | | | | | Number of | | | | | |
| | | | | | risk | | | | | |
| | | | IL1RN | TNFA | genetic | | | 95% CI | 95% CI | |
| Genetic marker | IL1B (−511) | IL1B (−3737) | (2018) | (−308) | units* | Frequency | OR | (Low) | (High) | P |
| SNP | | | | A/A, A/G | | 0.32 | 1.88 | 1.12 | 3.15 | 0.016 |
| Haplotype | C | T | | | | 0.42 | 1.52 | 0.94 | 2.45 | 0.085 |
| Diplotype | C/T | C/T | | | | 0.33 | 2.18 | 1.02 | 4.68 | 0.045 |
| Genetic units* | | T/T, C/T | C/C | A/A, A/G | 2 | 0.26 | 2.36 | 1.07 | 5.24 | 0.034 |
| | | | | | 3 | 0.03 | 5.09 | 1.10 | 23.49 | 0.037 |

*Number of risk genetic units is defined as the number of risk genotypes at IL1B (−3737), IL1RN (2018) and TNFA (−308) loci that are associated with lower response to anti-TNFα therapy.

Under this definition of response, one SNP (TNFA (−308)) and one diplotype pattern defined by the SNPs IL1B (−511) and IL1B (−3737) were identified as associated with lower response to anti-TNFα treatment (Table 5). Carriers of the TNFA (−308) genotype A/A or A/G, which comprises 32% of the study population, are 88% more likely to have lower response as compared to carriers of the G/G genotype (OR=1.88, p=0.016). Similarly, carriers of the diplotype pattern, defined by genotype C/T of the IL1B (−511) locus and genotype C/T of the IL1B (−3737) locus, have 1.18 fold increase in showing lower response to anti-TNFα therapy (OR=2.18, p=0.045). This group of people comprises 33% of the study population. The reference group consist of individuals with genotype C/C or C/T at the IL1B (−511) locus and C/C at the IL1B (−3737) locus.

A trend of association between response to anti-TNFα therapy and a haplotype pattern defined by IL1B (−511) and IL1B (−3737) was also identified (Table 5). Carriers of allele C of IL1B (−511) and allele T of IL1B (−3737) are more likely to have lower response to anti-TNFα therapy as compared to carriers of IL1B (−511) allele C and IL1B (−3737) allele C (OR=1.52, p=0.085). Carriers of the associated haplotype comprise 42% of the study population.

In single SNP analysis, IL1B (−3737) and TNFα (−308) were identified to be associated with lower response to anti-TNFα therapy (Tables 3 and 4). In addition, IL1RN (2018) was determined to have a trend of association with lower response to anti-TNFα therapy (Table 3). To assess the effect of multiple genetic risk factors on response to anti-TNFα therapy, we compared the response between individuals carrying multiple risk genotypes, or risk genetic units, at the IL1B (−3737), TNFA (−308) and IL1RN (2018) loci and those who carry no risk genotype (Table 5). Carriers of any two risk genetic units at the three loci have a 1.36-fold increase in not responding to anti-TNFα therapy (OR=2.36, p=0.034). Carriers of three risk genetic units have an even higher rate (4.09-fold increase) of not responding to the therapy (OR=5.09, p=0.037).

Anti-IL-1 and anti-TNFα therapies are commonly used for rheumatoid arthritis. Since the two types of medicines are not recommended to be administered at the same time, a test that can distinguish responsiveness to these medicines would be valuable. An IL1A polymorphism was previously shown to be associated with response to anti-IL-1 therapy in rheumatoid arthritis (Camp et al. (2005) Evidence of a pharmacogenomic response to interleukin-1 receptor antagonist in rheumatoid arthritis. Genes and Immunity 6: 467-471.). Individuals carrying genotype G/G at the IL1A (4845) locus are less responsive to the anti-IL-1 agent anakinra. To identify individuals who are less responsive to anti-IL-1 therapy but are more responsive to anti-TNFα therapy, we examined the association between anti-TNFα treatment response and inflammatory gene polymorphisms in a subgroup of people bearing the IL1A (4845) G/G genotype. In this subgroup, carriers of the T/T genotype at the IL1B (−3737) locus have a 1.2 fold increase in responsiveness to anti-TNFα therapy compared to individuals with the IL1B (−3737) C/T genotype (OR=2.20, p=0.039) (Table 6). Therefore, individuals bearing the IL1A (4845) G/G and IL1B (−3737) C/C genotype pattern, who comprise 13% of the study population, have lower response to anti-IL-1 therapy but higher response to anti-TNFα therapy.

TABLE 6
A composite genotype pattern associated with differential response
to anti-IL-1 and anti-TNFα therapies

| Genotype | | | | | | |
|---|---|---|---|---|---|---|
| IL1A | IL1B | | | 95% CI | 95% CI | |
| (4845) | (−3737) | Frequency | OR | (Low) | (High) | P* |
| G/G | C/C | 0.13 | 2.20 | 0.91 | 5.31 | 0.039 |

*one tailed test

### Example 2

### Questionnaire (Shown in Box Below)

We claim:

1. A method for determining the predisposition of a subject being treated for rheumatoid arthritis for an efficacious response to drugs that block TNFα biological activity comprising detecting a genetic pattern from one or a combination of multiple inflammatory genes selected from the group consisting of:
a) the gene for interleukin-1 alpha (IL1A);
b) the gene for interleukin-1 beta (IL1B);
c) the gene for interleukin-1 receptor antagonist (IL1RN);
d) the gene for interleukin-10(IL10);
e) the gene for tumor necrosis factor alpha (TNFA)
wherein the presence of said pattern indicates that said subject is predisposed to an efficacious response to anti-TNFα therapy.

2. A method for determining the predisposition of a subject being treated for rheumatoid arthritis for an efficacious response to drugs that block TNFα biological activity comprising detecting a pattern selected from the group consisting of:
a) two copies of IL1A (4845) allele T, two copies of IL1B(−511) allele C and two copies of IL1RN (2018) allele T;
b) two copies of IL1B (−511) allele T and two copies of IL1RN (2018) allele C; and
c) two copies of IL1B (−511) allele T, one copy of IL1RN (2018) allele T, and one copy of IL1RN (2018) allele C,
wherein the presence of said pattern indicates that said subject is predisposed to an efficacious response to anti-TNFα therapy.

3. The method of claim 2 wherein the anti-TNFα therapy is selected from the group consisting of etanercept, infliximab, and adalimumab.

4. The method of claim 2 wherein the efficacious genetic pattern is two copies of IL1A (4845) allele T, two copies of IL1B(−511) allele C, and two copies of IL1RN (2018) allele T.

5. The method of claim 2 wherein the efficacious genetic pattern is two copies of IL1B (−511) allele T and two copies of IL1RN (2018) allele C.

6. The method of claim 2 wherein the efficacious genetic pattern is two copies of IL1B (−511) allele T, one copy of IL1RN (2018) allele T, and one copy of IL1RN (2018) allele C.

7. A method for determining the predisposition of a subject being treated for rheumatoid arthritis for a reduced efficacious response to drugs that block TNFα biological activity comprising detecting a genetic pattern from one or a combination of multiple inflammatory genes selected from the group consisting of:
a) the gene for interleukin-1alpha (IL1A);
b) the gene for interleukin-1beta (IL1B);
c) the gene for interleukin-1 receptor antagonist (IL1RN);
d) the gene for interleukin-10 (IL10); and
e) the gene for tumor necrosis factor alpha (TNFA),
wherein the presence of said pattern indicates that said subject is predisposed to a reduced efficacious response to an anti-TNFα therapy.

8. The method of claim 7 wherein the anti-TNFα therapy is selected from the group consisting of etanercept, infliximab and adalimumab.

9. A method of claim 7 wherein the reduced efficacious response pattern is one copy of allele C and one copy of allele T at the locus IL1B(−511), and one copy of allele C and one copy of allele T at the locus IL1B(−3737).

10. A method of claim 7 wherein the reduced efficacious response pattern is one copy of allele C and one copy of allele T of IL1B(−511), and one copy of allele C and one copy of allele T of IL1B(−3737), and one or more of the genotypes selected from the group consisting of:
a) two copies of allele T of IL1RN (2018);
b) two copies of allele G at IL10 (−1082); and
c) one or two copies of allele G at TNFA(−308).

11. A method of claim 7 wherein the reduced efficacious response pattern is one or two copies of allele T at the locus IL1B (−3737).

12. A method of claim 7 wherein the reduced efficacious response pattern is one or two copies of allele A at the locus TNFA(−308).

13. A method of claim 7 wherein the reduced efficacious response pattern is 2 or more risk genetic units where the units are composed of:
a) one or two copies of allele T at IL1B (−3737); and
b) two copies of allele C at IL1RN (2018); and
c) one or two copies of allele A at TNFA (−308).

14. A method for determining the predisposition of a subject to differential response to drugs that block TNFα or IL-1 biological activity comprising detecting a genetic pattern from a combination of multiple inflammatory genes selected from the group consisting of:
a) the gene for interleukin-1alpha (IL1A); and
b) the gene for interleukin-1beta (IL1B),
wherein the presence of said pattern indicates that said subject is predisposed to a reduced efficacious response to an anti-IL1 therapy, but an increased response to an anti-TNFα therapy.

15. The method of claim 14 wherein the anti-IL-1 therapy is anakinra (IL-1Ra).

16. The method of claim 14 wherein the anti-TNFα therapy is selected from the group consisting of etanercept, infliximab and adalimumab.

17. The method of claim 14 wherein the pattern for reduced efficacious response to an anti-IL-1 therapy but an increased response to an anti-TNFα therapy is two copies of allele G at the locus IL1A (4845), and two copies of allele C at the locus IL1B(−3737).
